(19) United States
(12) Patent Application Publication
(10) Pub. No.: US 2014/0371321 A1
(43) Pub. Date: Dec. 18, 2014

(54) NOVEL PROCESS FOR CONCENTRATING SPILANTHOL AND/OR ISOMERS AND/OR HOMOLOGUES THEREOF

(71) Applicant: ROBERTET SA, Grasse (FR)

(72) Inventors: Andre Casazza, Grasse (FR); Karine Kormann, Valbonne (FR)

(21) Appl. No.: 14/374,279

(22) PCT Filed: Jan. 18, 2013
(86) PCT No.: PCT/FR2013/050121

§ 371 Date: Jul. 24, 2014

(30) Foreign Application Priority Data
Jan. 24, 2012 (FR) 12 50657

(51) Int. Cl. A61K 31/16; A61K 47/02; A61K 47/08; A61K 8/31; A01N 37/20; A61K 8/19; A61K 8/33; A61K 8/42; A61K 36/28; A61K 47/06

(52) U.S. Cl. CPC A61K 31/16; A61K 36/28; A61K 47/02; A61K 47/08; A61K 47/06; A01N 37/20; A61K 8/19; A61K 8/33; A61K 8/42; A61K 8/31
USPC 514/627

(57) ABSTRACT

A subject of the invention is a process for concentrating the spilanthol contained in a composition, comprising the formation of an addition compound of spilanthol with a strong acid, then the hydrolysis of said addition compound.

The process according to the invention makes it possible to easily obtain a composition which may comprise 100% either isolated actual spilanthol ((2E,6Z,8E)-N-(2-methyl propyl)deca-2,6,8-trienamide), or a mixture of actual spilanthol with the isomers andor homologues thereof that may accompany it.

The invention lies within the field of chemistry, particularly in the field of processes for concentrating a product contained in a composition. More particularly the invention relates to a novel process for concentrating amide compounds and very particularly spilanthol, isomers and homologues thereof.

Jamba extracts (tinctures, concretes, absolutes, essential oil, oleoresins, distillates, etc.) can be obtained from all parts of the plants of the genus Spilanthes of the family of the Compositae (Spilanthes acmella or acmelia, Spilanthes ciliate, Spilanthes acmella var. Murr., Spilanthes acmella var. oleracea, Spilanthes americana, Spilanthes nervosa, Spilanthes leiocarpa, Spilanthes paraguyensis, Spilanthes chamaecaula, Spilanthes macraei, Spilanthes oleracea, Spilanthes oppositifolia, Spilanthes alba, Spilanthes calvus, Spilanthes mauritiana, Spilanthes costata, Spilanthes urens, Spilanthes anactina, Spilanthes repens, Spilanthes beccabunga, preferably Spilanthes acmella var. oleracea (also referred to as Spilanthes acmella oleracea or Acmella oleracea).

The inflorescences of the plants of the genus Spilanthes, composed of yellow flowers, and the leaves have a pungent taste. in the Brazilian State of Pare, the plants andor extracts are fundamental ingredients in traditional cookery as well as in traditional medicine.

The properties of these plants are attributed mainly to spilanthol, also called affinine, or (2E,8Z,8E)-N-(2-methyl propyl)deca-2,6,84rienamide [CAS 25394-57-4] (also referred to as “actual spilanthol” in the present text), which is an isobutyl amide having the following formula:

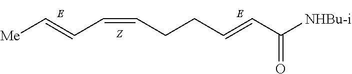

Because of its structure this compound can have numerous isomers.

It is moreover known (Martin R. and Becker H., Phytochemistry, vol. 23, No 8, pp. 1781-1783, 1984) that the plant extracts comprising actual spilanthol also comprise homologues of said actual spilanthol including for example those which correspond to Formula I below

R2—CH2—CH═CH—CH═CH—CH2—CH2—CHA—CHA—CO—NH—R1

in which

R1 can represent a —(CH2)—CH(CH3)2 radical, or a —(CH2)—CH(CH3)—CH2—CH3 radical, or a —CH2—CH2—C6H5 radical; and

R2 can represent a hydrogen atom or an —O—CO—CH2—CH(CH3)2 radical, or an —O—CO—CH═CH(CH3)2 radical and

A represents a hydrogen atom or, together with the carbon atoms that bear them forms a double bond.

By way of example, the following may be mentioned as homologues of actual spilanthol: 2-methyl butyl amide spilantic acid (R1=—(CH2)—CH(CH3)—CH2—CH3, R2═H, and A form a double bond), 2-phenylethyl amide spilantic acid (R1=—CH2—CH2—C6H5, R2═H and A form a double bond), 10-hydroxyspilanthollsovalerate (R1=—(CH2)—CH(CH3)2, R2 =—O—CO—CH2—CH(CH3)2 and A form a double bond), 10-hydroxyspilanthol-(3-methyl acrylate) (R1=—(CH2)—CH(CH3)2, R2=—O—CO—CH═CH(CH3)2 and A form a double bond), or also hydrospilanthol or 2,3-dihydrospilanthol (R1=—(CH2)—CH(CH3)2, R2 and A═H).

As a general rule, in the plant extracts which contain it, (2E,6Z,8E)-N-(2-methyl propyl)deca-2,6,8-trienamide (called “actual spilanthol” elsewhere in the present text) is quantitatively in the vast majority compared with these homologues.

Thus in the present text, unless otherwise indicated, the use of the term “spilanthol” will cover both isolated actual spilanthol ((2E,6Z,8E)-N-(2-methyl propyl)deca-2,6,8-trienamide), and mixtures of actual spilanthol with the isomers andor homologues thereof that may accompany it.

Spilanthoi is known to have numerous applications.

It is used, for example in medicine as an analgesic for toothache, for the treatment of aphthae and herpes, for stomatitis and throat infections, as a sialagogue or also as a cicatrizant.

Insecticidal activities have been described, such as in particular larvicidal action against the larvae of Culex quinquefasciatus, against Aedes aegyptii, against tics, cockroaches or also mites.

Spilanthol has also been described as an effective antimicrobial agent particularly against Proteus mirabilis, Pseudomonas aeruginosa, Staphylococcus aureus or Candida albicans, as a fungistatic and fungicide for example against Aspergillus spp, or also as an antimutagenic agent.

Spilanthol is also used in the manufacture of anti-wrinkle cosmetic compositions or in cosmetic compositions for stressed skin.

Other properties of spilanthol are also widely used, such as for example its ability to provide a tingling sensation for example to prolong the fizzy sensation particular to carbonated drinks, or a refreshing sensation in cosmetic compositions. They are also found in numerous compositions of sweets or chewing-gums.

Spilanthol is therefore a widely-used compound.

It is notable that apart from Jambu oleoresin, other extracts of other plants are known to comprise spilanthol. in this connection mention may be made of the plants of the genus Heliopsis of the family of the Compositae, for example the species Heliopsis buphthalmoides, Heliopsis gracilis, Heliopsis helianthoides, Heliopsis longipes, Heliopsis oppositifolia, or also Heliopsis parvifolia.

The preparation of spilanthol can be either purely synthetic (for example such as that described in WO12011007807) or also by extraction from Jambu as described for example in the Patent Application U.S. 20088/0171003.

But in general it is difficult to obtain easily, industrially and at the lowest possible cost, compositions with a high concentration of spilanthol or even compositions containing only 100% spilanthol within the meaning of the term defined previously.

Thus there remains a need for a spilanthol production method which will overcome the known drawbacks (difficulties, complexities, length of time, cost, poor yield, etc.) of the methods described in the prior art (without of course calling the qualities thereof into question).

The invention therefore relates to a novel process for concentrating spilanthoi (actual spilanthol, its amide-type isomers and homologues, such as for example those corresponding to Formula I), that is simple, fast, inexpensive and easily industrialized which makes it possible, starting from any composition comprising spilanthol in any concentration, to obtain a composition comprising 100% of a whole constituted by actual spilanthol and optionally amide isomers andor homologues of said actual spilanthol, said composition comprising at least 90% actual spilanthol, advantageously 95%.

According to the invention, said composition obtained at the end of the process can be in any conceivable form, but is generally presented in the form of a thick, oily liquid. Any method making it possible to determine the nature of the compounds contained in said composition can be used. The technique of high performance liquid chromatography (HPLC) or that of gas chromatography (GC) with internal calibration according to the standard techniques commonly used in chemistry, as for example described in Chemical Analysis, Modern Instrumental Methods and Techniques (Francis & Annick Rouessac, 2000, John Wiley & Sons, Ltd, publishers) will be mentioned as examples.

Thus a subject of the invention is a novel process for concentrating spilanthol, contained in a composition, comprising the formation of an addition derivative of the spilanthoi, as well as, optionally, the amide-type isomers and homologues thereof, with a strong acid (addition step), an addition derivative which can then be hydrolyzed in order to regenerate said spllanthol, as well as, optionally, the amide-type isomers and homologues thereof, in the form of composition comprising actual spilanthol, as well as, optionally, the amide-type isomers and homologues thereof.

According to the invention, said composition comprising spilanthol (within the meaning defined previously, namely actual spilanthol, amide-type isomers and homologues), can be any composition, whether natural or synthetic, which contains spllanthol in any quantity whatever that is to be concentrated.

By “natural composition comprising spilanthol” is meant a composition obtained from a natural product such as for example a plant, among which those of the family of the Compositae, advantageously those of the genera Spilanthes and Heliopsis will be mentioned.

In this connection the natural compositions comprising spilanthol can be any type of extract irrespective of the operating method from which it results (extraction, distillation). In this connection the aqueous extracts or the non-aqueous extracts, particularly those obtained using solvents will be mentioned. The oleoresins, essential oils, absolutes, concretes or also extracts obtained using hydrofluorocarbon (HFC) may be mentioned.

According to the invention, the natural compositions comprising spilanthol can be any type of extract of all the parts of the plants in question.

By “synthetic composition comprising spilanthol” is meant any composition comprising spilanthol, obtained otherwise than by extraction or distillation from a natural product, such as for example after a chemical synthesis or a hemi-synthesis from a natural precursor.

According to the invention, by “strong acid” is meant an acid which, in aqueous solution, breaks down completely into an H+ proton and a very weak base known as a conjugate base of the acid.

The following may be mentioned as strong acids that can be used according to the invention: hydrochloric acid (HCl), concentrated sulphuric acid (92-98% H₂SO₄), phosphoric acids, particularly orthophosphoric acid (H₃PO₄), oxalic acid, particularly anhydrous oxalic acid (C₂H₂O₄), nitric acid (HNO₃), hydriodic acid (Hl), hydrobromic acid (HBr), perchloric acid (HClO₄), chioric acid (HClO₃), permanganic acid (HMnO₄), manganic acid (H₂MnO₄), fluoroantimonic acid (HF·SbF₅), magic acid (HSO₃F·SbF₅), methanesulphonic acid (CH3SO3H), trifluoromethanesulphonic acid or triflic acid (HSO₃CF₃), fluorosulphuric acid or fluorosulphonic acid (HSO₃F), naphthalene-l-sulphonic acid (C₁₀H₇SO₃H), naphthalene-1,5-disuiphonic acid (C₁₀H₆(SO₃H)₂), disulphuric acid or oleum (H₂S2O₇), or also para-toluene sulphonic acid (PTSA). Preferably, according to the invention it is possible to use a strong acid chosen from concentrated sulphuric acid (H₂SO₄), the phosphoric acids, particularly orthophosphoric acid (H₃PO₄), oxalic acid, particularly anhydrous oxalic acid (C₂H₂O₄), very preferably, concentrated sulphuric acid.

It is therefore understood that in the first step of the process according to the invention, an addition derivative of spilanthol contained in a composition has been obtained by acidification of said composition. This addition derivative corresponds to an addition complex of the strong acid with the basic function of the amide. The second step of the process according to the invention will allow the regeneration of the spilanthol. This step can be carried out by the addition of a base to the medium comprising the addition derivative obtained in the first step.

According to the invention. the base that can be used in the second hydrolysis step can be sodium carbonate, sodium bicarbonate, lime (Ca(OH)₂), ammonium hydroxide, soda, lithium hydroxide (LiOH), potassium hydroxide (KOH), or also barium hydroxide (Ba(OH)₂). Preferably according to the invention, the base that can be used in the second hydrolysis step can be sodium carbonate or sodium bicarbonate.

According to the invention, the first step of the spilanthol isolation process can be carried out in an anhydrous medium. This means that, in the first step of the process, at least the composition comprising spilanthol is mixed with an acid-inert solvent and that optionally the strong acid used is also mixed with an acid-inert solvent.

Another advantage of carrying out the first step of the spilanthol isolation process in an anhydrous medium is obtaining an insoluble complex that can be easily separated from the reaction medium by simple settling.

In general, commercially-available “pure” reagents such as the strong acids listed previously are perfectly suitable without having to be subjected to drying treatment. But if this proves necessary, these reagents can be mixed, before reaction, with at least one acid-inert solvent, allowing their drying. It may therefore be necessary according to the invention for the different reagents involved in the first step of the process to be anhydrous or considered as such. Very advantageously, the different reagents involved in the first step of the process may comprise no more than 0.5% water.

Thus a subject of the invention is a process for concentrating the spilanthol contained in a composition, comprising the formation of an addition derivative of spilanthol with a strong acid, said composition comprising the spilanthol being rendered anhydrous by mixture with an acid-inert solvent, then hydrolysis of said addition compound obtained previously.

Advantageously he strong acid used according to the invention for the formation of the addition derivative can be rendered anhydrous by mixture with an acid-inert solvent.

Thus according to the invention, the process can comprise the following steps: a first step of putting a composition comprising spilarithol in anhydrous solution by mixing said composition comprising spilanthol with an acid-inert solvent;

a second addition step by mixing said anhydrous solution obtained in the first step with a strong acid, itself advantageously also in solution in an anhydrous medium in order to obtain an oily product;

a third step in which said oily product obtained in the second step can be brought into the presence of a base in order to obtain a spilanthol solution.

According to the invention, the spilanthol solution obtained at the end of the process can be used immediately or undergo any operations necessary for the removal of any residues of acid-inert solvent that it may still contain, for its drying, for its filtration and for changing its appearance.

According to the invention, said useable acid-inert soivents can be selected from the ethers such as for example diethyl ether, di-isopropyl ether, methyl tert-butyl ether (MTBE), tetrahydrofuran (THF), methyl tetrahydrofuran (MeTHF), dioxane, dimethoxyethane (DME), anisole (methoxybenzene), crown ethers or also polyethylene glycol (PEG),

the hydrocarbons such as for example

the linear or branched, substituted or unsubstituted alkanes, comprising between 5 and 8 carbon atoms, such as for example n-pentane, isopentane, neopentane, n-hexane, 2-methyl pentane, 3-methyl pentane, 2,2-dimethyl butane, 2,3-dimethyl butane, n-heptane, 2-methyl hexane, 3-methyl hexane or its racemic mixture of R-3-methyl hexane and 5-3-methyi hexane, 2,2-dimethyl pentane, 2,3-dimethyl pentane or its racemic mixture of R-2,3-dimethyl pentane and S-2,3-dimethyl pentane, 2,4-dimethyl pentane, 3,3-dimethyl pentane, 3-ethyl pentane, 2,2,3-trimethyl butane, n-octane, 2-methyl heptane, 3-methyl heptane or its racemic mixture of (R)-3-methyl heptane and (S)-3-methyl heptane, 4-methyl heptane, 3-ethyl hexane, 2,2-dimethyl hexane, 2,3-dimethyl hexane or its racemic mixture of (R)-2,3-dimethyl hexane and (S)-2,3-dimethyl hexane, 2,4-dimethyl hexane or its racemic mixture of (R)-2,4-dimethyl hexane and (S)-2,4-dimethyl hexane, 2,5-dimethyl hexane, 3,3-dimethyl hexane, 3,4-dimethyl hexane or its racemic mixture and its diastereoisomers (meso compound) (R,R)-3,4-dimethyl hexane, (5,5)-3,4-dimethyl hexane, (3R,4S)-3,4-dimethyl hexane or (3S,4R)-3,4-dimethyl hexane, 3-ethyl-2-methyl pentane, 3-ethyl-3-methyl pentane, 2,2,3-trimethyl pentane or its racemic mixture of (R)-2,2,3-trimethyl pentane and (S)-2,2,3-trimethyl pentane, 2,2,4-trimethyl pentane or isooctane, 2,3,3-trimethyl pentane, 2,3,4-trimethyl pentane, or also 2,2,3,3-tetramethyl butane;

the substituted or unsubstituted aromatic cycloalkanes and compounds, comprising between 5 and 8 carbon atoms, such as for example cyclopentane, cyclohexane, cycloheptane, cyclooctane, xylene, benzene, or also toluene, preferably benzene, and their derivatives or isomers;

the ketones such as for example acetone, methyl ethyl ketone, methyl isobutyl ketone (MIBK), pinacolone, diethyl ketone (3 pentanone), diisopropyl ketone, or also 2,4 dimethyl 3 pentanone, preferably methyl isobutyl ketone.

Preferably according to the invention it is possible to use diethyl ether, dilsopropyl ether, methyl tert-butyl ether (MTBE), methyl tetrahydrofuran (MeTHF), benzene, toluene or also methyl isobutyl ketone (MIBK), very preferably diisopropyl ether.

According to the invention, the step of putting a composition comprising spilanthol in anhydrous solution can be carried out by mixing said composition comprising spilanthol with said acid-inert solvent in order to obtain a final solution comprising said composition comprising spilanthol in a proportion comprised between 20 and 50% (weight/volume), preferably in a proportion comprised between 30 and 40% (weight/volume).

According to the invention, when in the addition step the strong acid used is itself in solution in an anhydrous medium, said anhydrous strong acid solution can be obtained by mixing said strong acid with said acid-inert solvent in order to obtain a final solution comprising strong acid in a proportion comprised between 2 and 15% (volume/volume), preferably in a proportion comprised between 3 and 14% (volume/volume).

According to the invention, in the hydrolysis (or regeneration) step, said base can be added to said oily product obtained in the second step in a volume representing from 25% to 75% of the volume of said oily product, advantageously representing from 40 to 60% of the volume of said oily product.

According to the invention, said base used in the hydrolysis (or regeneration) step can be in solution, advantageously aqueous, in a proportion comprised between 5 and 15% advantageously 10%.

According to the invention, the addition step by mixing said solution comprising anhydrous spilanthol with a strong acid can be carried out by incorporation of one of the solutions in the other, advantageously of the acid solution in the solution comprising spilanthol, under stirring, at ambient temperature (approximately 25° C. +/−5° C.). When the solutions are mixed, said mixture can be maintained under stirring for a time comprised between 1 and 20 minutes, advantageously between 1 and 5 minutes. At the end of the stirring, said oily product formed is left to settle before recovering it in order to carry out the regeneration step making it possible to recover the spilanthol that it comprises.

The hydrolysis (or regeneration) step can be carried out by mixing said oily product with the base under stirring for a time comprised between 5 and 120 minutes, advantageously between 5 and 30 minutes. It is possible during this phase of the process to verify the complete consumption of the acid by measuring the pH of the solution obtained which, when it is comprised between 7 and 10, advantageously between 7.5 and 8.5, indicates that the acid has been completely neutralized.

The solution is then left to settle, followed by recovery of the organic phase which can then undergo any operations necessary for removal of the residues of said acid-inert solvent that it may still comprise, for its drying, its filtration and its concentration. Thus it is possible for example to carry out a step of washing the organic phase. This step is optional; a person skilled in the art will know to carry out washing if needed, The solution obtained can comprise 100% either of isolated actual spilanthol ((2E,6Z,8E)-N-(2-methyl propyl)deca-2,8,8-trienamide), or a mixture of actual spilanthol with the isomers andor homologues thereof that may accompany it.

Depending on the strong acid used, it is possible that in the step of formation of an addition derivative of spilanthol with a strong acid, after mixture of the composition comprising the spilanthol with the strong acid, the solution obtained is not presented in the insoluble form. It may then be necessary to subject this solution to any steps necessary so that two phases separate. A person skilled in the art will have no problem carrying out these additional steps which are perfectly normal in chemical synthesis. For example said residual acid-inert solvent may be evaporated until the phases separate, for example using a rotary evaporator. The residue then obtained can then be taken up and washed in a solvent such as for example hexane, cyclohexane or also diisopropyl ether before being treated with said base in order to carry out the regeneration step.

Other advantages and properties of the process according to the invention will become apparent on reading the following examples, without however considering these as limitative of the invention.

It is significant that for all the examples described hereafter the chromatographies carried out in order to ensure the nature of the compounds contained in the oily product obtained at the end of the process can be superimposed and only have peaks corresponding to actual spilanthol, in a large majority, and to the homologues thereof.

### EXAMPLES

### Example 1

### Concentration of the Spilanthol Contained in a Jambu Extract

55.0 g of Jambu absolute titrated at 36% actual spilanthol is solubilized in 165 mL of ethyl ether. At the same time 8.0 of concentrated sulphuric acid is dissolved in 240 mL of ethyl ether.

Under thorough stirring, the acid solution is poured into the Jambu solution. A change in coloration appears. The mixture is maintained under stirring for another minute or two and then left to settle. Rapidly, a black oily phase collects at the bottom of the container.

This black oily phase is then drawn off and stirred in the presence of 100 mL of ethyl ether, then again left to settle.

The black oily phase is taken up in 300 mL of ethyl ether and stirred in the presence of 175 of sodium carbonate at 10% in water.

The pH measurement makes it possible to verify that all the acid has been well neutralized (expected pH 8.0) and the settling continued.

The ethereal solution now containing the spilanthol is then dried, filtered and concentrated in a rotary evaporator to provide 21.0 g of an oil comprising 100% of a mixture containing at least 90% actual spilanthoi accompanied by the amide isomers and homologues thereof.

### Example 2

### Concentration of the Spilanthol Contained in a Jambu Extract

60.0 g of Jambu oleoresin titrated at 60% actual spilanthol is solubilized in 180 mL of diisopropyl ether. At the same time 9.0 mL of concentrated sulphuric acid is solubilized in 225 mL of dilsopropyl ether.

The reaction is carried out in exactly the same way as for Example 1, in order to obtain 39.0 g of an oil comprising 100% of a mixture containing at least 90% actual spilanthol accompanied by the amide isomers and homologues thereof.

### Example 3

### Concentration of the Spilanthol Contained in a Jambu Extract

60.0 g of Jambu oleoresin titrated at 60% actual spilanthol is solubilized in 180 mL of diisopropyl ether and 17.0 g of crystallized orthophosphoric acid (100% acid) is suspended in 170 mL of diisopropyl ether.

The oleoresin solution is poured into the suspension of acid and the mixture is stirred for about ten minutes, until the acid completely disappears and a yellow solution is obtained.

The yellow solution obtained is then concentrated in the rotary evaporator. Approximately 250 mL of dlisopropyi ether is then evaporated using a rotary evaporator under a pressure of 25 hPa and recovered for subsequent use. The residue is taken up in 300 mL of hexene and stirred for a few minutes then left to settle. A brick-red oily phase collects at the bottom of the container. This oily phase is then drawn off and stirred in the presence of 100 mL of hexane. The mixture is left to settle.

The brick-red oily phase is taken up in the 250 mL of dilsopropyl ether distilled above and treated as in the case of Example 1 in order to finally produce 39.0 g of an oil comprising 100% of a mixture containing at least 90% actual spilanthol accompanied by the amide isomers and homologues thereof.

### Example 4

### Concentration of the Spilanthol Contained in a Jambu Extract

25.0 g of Jambu oleoresin containing 60% actual spilanthol is solubilized in 75 ml. of benzene, and 3.75 mL of concentrated sulphuric acid is emulsified in 75 mL of benzene under vigorous stirring.

The emulsion is poured into the oleoresin solution under vigorous stirring.

A change in coloration appears and when the stirring stops an insoluble oily product is deposited at the bottom of the container.

After settling and washing with the benzene of the oily phase the spilanthol is regenerated by neutralization with sodium carbonate and extraction with benzene.

The benzene phase is then dried, filtered and concentrated in a rotary evaporator to provide 13.5 g of an oil comprising 100% of a mixture containing at least 90% actual spilanthol accompanied by the amide isomers and homologues thereof.

### Example 5

### Concentration of the Spilanthol Contained in a Jambu Extract

25.0 g of Jambu oleoresin titrated at 60% actual spilanthol is solubilized in 75 mL of dilsopropyl ether, and 6.5 g of anhydrous oxalic acid is dissolved in 65 mL of the same solvent.

The two solutions are mixed then the same treatment as that carried out in Example 4 is applied to the mixture.

14.0 g of an oil comprising 100% of a mixture containing at least 90% actual spilanthol is then obtained, accompanied by the amide isomers and homologues thereof,

### Example 6

### Concentration of the Spilanthol Contained in a Jambu Extract

25.0 g of Jambu oleoresin titrated at 60% actual spilanthoi in solution in 75 mL of methyl isobutyl ketone (MIBK) are mixed under stirring with a solution of 3.75 mL of concentrated sulphuric acid in 75 mL of MIBK.

The medium darkens but no phase separation appears.

The same treatment as that applied in Example 5 is applied to the mixture.

15.0 g of an oily solution comprising 100% of a mixture containing at least 90% actual spilanthol is then obtained, accompanied by the amide isomers and homologues thereof.

### Example 7

### Concentration of the Spilanthol Contained in a Jambu Extract

60.0 g of Jambu absolute titrated at 40% actual spilanthol is solubilized in 180 of methyl tert-butyl ether (MTBE) and 9.0 mL of concentrated sulphuric acid is solubilized in 180 mLofN1T8E also.

A reaction occurs upon mixture of the two solutions but no insoluble product as in Examples 1 to 3 appears.

Approximately 250 mL of MTBE is then evaporated using a Büchi rotary evaporator under a pressure of 25 hPa.

The residue that appears is then taken up in 360 mL of hexane under stirring for 5 minutes then left to settle. A black oily phase collects at the bottom of the container. The black oily phase is then drawn off and stirred in the presence of 130 mL of hexane. The mixture is left to settle.

The black oily phase is then taken up in 250 mL of hexane and treated as in the case of Example 1 in order to finally produce 24.0 g of an oil comprising 100% of a mixture containing at least 90% actual spilanthol accompanied by its isomers and homologues.

### Example 8

### Concentration of the Spilanthol Contained in a Jambu Extract on a Large Scale

360 g of Jambu oleoresin containing 60% actual spilanthol is solubilized in 1080 mL of MTBE. and 54 mL of concentrated sulphuric acid is sclubilized in 540 mL of MTBE (this last dissolution is very exothermic; it is carried out in an ice bath so that the temperature remains below 20° C.).

The two solutions are then rnIxed and an insoluble product in the form of an oily phase appears.

The dark oily phase is left to settle for at least ten minutes. The oily phase is then drawn off and washed with 500 mL of MTBE.

500 mL of fresh MTBE is added to the re-settled oily phase and treated with approximately 1500 mL of an aqueous solution of sodium carbonate at 7.5% in order to obtain a final pH of the solution of 8.

After settling and re-treating the organic phase (TBE), a second extraction is carried out on the aqueous phase with 250 mL of MTBE.

Settling is slow in both cases.

The combined organic phases (MTBE) are washed with 400 mL. of 25% sodium chloride (NaCl) solution.

Slow settling is then continued in order to recover an organic phase (MTBE) which, after drying, filtration and evaporation to dryness, makes it possible to obtain 208 g of an oil comprising 100% of a mixture containing at least 90% actual spilanthol accompanied by the amide isomers and homologues thereof.

This example shows that the process according to the invention can easily be adapted to an industrial scale.

### Example 9

### Concentration of the Spilanthol Contained in a Jambu Extract on a Large Scale

450 g of Jambu oleoresin containing 60% actual spilanthol is solubilized in 1350 mL (i.e. 3 vol.) of di-isopropyl ether, and 70 mL of concentrated sulphuric acid is solubilized in 1350 of di-isopropyl ether while cooling in an ice bath, The dissolution of the concentrated sulphuric acid is exothermic, the temperature rising from +7 to +28° C.

Under thorough stirring, the acid solution is then poured into the Jambu solution. A yellow precipitate, becoming brownish, is formed.

The stirring is then maintained for 2 minutes then the mixture is left to settle for at least 10 minutes.

A brown oily phase is deposited at the bottom of the container.

The oily phase, which is fairly thick, is drawn off and stirred for one minute in the presence of 675 mL of di-isopropyl ether, then a new settling phase is continued for at least 10 minutes.

The oily phase thus washed is taken up in 900 mL of di-isopropyl ether then treated under thorough stirring with a solution of 150 g of sodium carbonate (Na₂CO₃) in 750 mL of water.

Verification that all the acid has been well neutralized (ph approximately 8) is carried out with paper.

The organic phase is dried, filtered and concent a ed using a BCichi rotary evaporator under a pressure of 25 hPa.

In this way 292.3 g (yield 65.0%) of an oil comprising 100% of a mixture containing at least 90% actual spilanthol accompanied by the amide isomers and homologues thereof is obtained.

### Example 10

### Concentration of the Spilanthol Contained in a Jambu Extract on an Industrial Scale

In a 2-L Erlenmeyer flask, 150 mL of 92% sulphuric acid (industrial acid) is poured by portions, under stirring, into 1100 mL (approximately 7.5 volumes) of di-isopropyl ether, without exceeding +15° C., using a cooling bath,

During this time, in a 5-L vessel, a solution of 750 g of Jambu oleoresin containing 61% of spilanthol in 2250 mL (i.e. 3 volumes) of di-isopropyl ether is prepared.

Remaining at +15°, the acid solution is poured into the well-stirred Jambu solution. A yellow oil separates, which becomes brownish. The mixture is stirred for another 3 to 5 minutes then left to settle for 10 to 15 minutes, The two phases are very distinct, the interface clearly defined.

During settling, a solution of 300 g of sodium carbonate in 1875 mL of water is prepared.

The brown oil is drawn off in an ampoule and washed with 1500 mL, of di-isopropyl ether. The mixture is left to settle. The oil has darkened.

The washed oil is suspended in 1500 mL of fresh di-isopropyl ether. Under thorough stirring, the solution of carbonate is added slowly (15 minutes). The mixture is again stirred for 5 minutes then left to settle: the di-isopropyl ether phase is light brown, the aqueous phase is pale yellow, presence of crude ethyl alcohol. The pH of the aqueous phase is verified (expected pH 8). The aqueous phase is drawn off and the organic phase is concentrated in the rotary evaporator, under a pressure of 25 hPa, in a water bath at 60° C. 466.8 g of an oil comprising 100% of a mixture containing at least 90% actual spilanthol accompanied by the amide isomers and homologues thereof are obtained.

A second extraction of the aqueous phase (750 mL of di-isopropyl ether) provides no more than 0.3 g, it is therefore virtually useless.

This example shows that the process can be adapted to an industrial scale. Furthermore, it shows that it is possible to carry out the process according to the invention with 92% sulphuric acid, which is the sulphuric acid found in commerce, and which is less expensive than concentrated, for example 98%, sulphuric acid.

1. Process for concentrating spilanthol contained in a composition, comprising:
a. forming an addition derivative of spilanthol with a strong acid, said composition comprising the spilanthol being rendered anhydrous by mixture with an acid-inert solvent, then
b. the performing hydrolysis of said addition compound.

2. Process according to claim 1, wherein the strong add is rendered anhydrous by mixture with an acid-inert solvent.

3. Process according to claim 1, wherein the strong acid is selected from the group consisting of hydrochloric acid, concentrated sulphuric acid, the phosphoric acids, particularly orthophosphoric acid, oxalic acid, particularly anhydrous oxalic acid, nitric acid, hydriodic acid, hydrobromic acid, perchioric acid, chioric acid, permanganic acid, manganic acid, fluoroantimonic acid, magic acid, rnethanesulphonic acid, trifluoromethanesuiphonic acid or triflic acid, fluorosulphuric acid or fluorosulphonic acid, naphthalene-l-sulphonic add, naphthalene-1,5-disulphonic acid, disulphuric acid or oleum, and para-toluene sulphonic acid.

4. Process according to claim 1, wherein the hydrolysis step is carried out by mixing said addition compound with a base.

5. Process according to claim 4, wherein the base is sodium carbonate, sodium bicarbonate, lime, ammonium hydroxide, soda, lithine, potassium hydroxide, or also barium hydroxide.

6. Process according to claim 1, wherein said acid-inert solvent is an ether, a hydrocarbon or a ketone.

7. Process according to claim 6, wherein said ether is selected from the group consisting of diethyl ether, di-isopropyi ether, methyl tent-butyl ether, tetrahydrofuran, methyl tetrahydrofuran, dioxane, dimethoxyethane, anisole, crown ethers and polyethylene glycol.

8. Process according to claim 6, wherein said hydrocarbon is linear or branched, substituted or unsubstituted alkanes, comprising between 5 and 8 carbon atoms, or the substituted or unsubstituted aromatic cydoalkanes and compounds, comprising between 5 and 8 carbon atoms,

9. Process according to claim 6, wherein is selected from the group consisiting of acetone, methyl ethyl ketone, methyl isobutyl ketone, pinacolone, diethyl ketone, diisopropyl ketone, and 2,4 dimethyl 3 pentanone.

10. Process according to claim 6, wherein said acid-inert solvent is selected from the group consisting of diethyl ether, di-isopropyl ether, methyl tert-butyl ether, methyl tetrahydrofuran, benzene, toluene and methyl isobutyl ketone.

11. Process according to claim 1, wherein it comprises:
a. a first step of putting in anhydrous solution a composition comprising spilanthol by mixing said composition comprising spilanthol with an acid-inert solvent;
b. a second addition step by mixing the anhydrous solution obtained in the first step with a strong acid, itself advantageously also in solution in an anhydrous medium in order to obtain an oily product; and
c. a third step of placing the oily product obtained in the second step, itself advantageously in an anhydrous medium, which can be brought into the presence of a base in order to obtain a spilanthol solution.

12. Process according to claim 11, wherein the step of putting in anhydrous solution a composition comprising spilanthol, is carried out by mixing said composition comprising spilanthol with said acid-inert solvent in order to obtain a final solution comprising said composition comprising spilanthol in a proportion comprised between 20 and 50% (weight/volume),

13. Process according to claim 2, wherein the anhydrous solution of strong acid is obtained by mixing said strong acid with an acid-inert solvent in order to obtain a final solution comprising strong acid in a proportion comprised between 2 and 15% (volume/volume).

14. Process according to claim 11, wherein in the third step, the base is added to the oily product obtained in the second step, in a volume representing from 25% to 75% of the volume of said oily product.

15. Process according to claim 11, wherein the base used in the hydrolysis step is in solution, advantageously aqueous, in a proportion comprised between 5 and 15%.
